(19) United States
(12) Patent Application Publication
(10) Pub. No.: US 2026/0351409 A1
(43) Pub. Date: Nov. 5, 2026

(54) USING A MACHINE LEARNING MODEL PRETRAINED WITH UNLABELED TRAINING DATA TO GENERATE INFORMATION CORRESPONDING TO CARDIAC DATA SENSED BY A MEDICAL DEVICE

(71) Applicant: Medtronic, Inc., Minneapolis, MN (US)

(72) Inventors: Brandon D. Stoick, Maple Lake, MN (US); Tarek D. Haddad, Minneapolis, MN (US)

(21) Appl. No.: 19/487,318

(22) PCT Filed: May 2, 2024
(86) PCT No.: PCT/IB2024/054274

§ 371 Date: Nov. 24, 2025

Related U.S. Application Data
(60) Provisional application No. 63/505,202, filed on May 31, 2023.

(51) Int. Cl. G16H 50/20; G06N 20/00; G16H 40/67

(52) U.S. Cl. CPC G16H 50/20; G06N 20/00; G16H 40/67

(57) ABSTRACT

A medical device system includes a medical device comprising one or more electrodes and configured to generate cardiac data based on a cardiac signal sensed via the one or more electrodes; and a memory configured to store: a machine learning model; a first plurality of training datasets; and a second plurality of training datasets. Additionally, the medical device system includes processing circuitry in communication with the memory, wherein the processing circuitry is configured to: receive, from the medical device, the cardiac data; and generate information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.

This application claims the benefit of U.S. Provisional Patent Application Ser. No. 63/505,202, filed May 31, 2023, the entire content of which is incorporated herein by reference.

### TECHNICAL FIELD

This disclosure is directed to medical devices and, more particularly, to systems and methods for monitoring patient conditions.

### BACKGROUND

A variety of devices are configured to monitor physiological signals of a patient. Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

In some cases, such devices are configured to detect health events, such as episodes of cardiac arrhythmia or worsening of heart failure, based on the physiological signals. Example arrhythmia types include asystole, bradycardia, ventricular tachycardia, supraventricular tachycardia, wide complex tachycardia, atrial fibrillation, atrial flutter, ventricular fibrillation, atrioventricular block, premature ventricular contractions, and premature atrial contractions. The devices may store ECG and other physiological signal data collected during a time period including an episode as episode data. The devices may also store episode data quantifying the episodes, e.g., number and/or duration of episodes. The medical device may also store ECG and other physiological data for a time period as episode data in response to user input, e.g., from the patient or a caregiver.

### SUMMARY

In general, the disclosure is directed to techniques for monitoring patient conditions based on biometric data collected by a medical device. Some systems may be configured to identify a current status of a patient condition and a past status of the patient conditions. Additionally, or alternatively, some systems may be configured to predict a future status of a patient condition based on biometric data collected by a medical device, even when the biometric data represents data up to a present time. It may be advantageous for a system to predict a future status of a patient condition so that the system is able to control therapy delivered to a patient based on the predicted future status or change a medication regimen administered to the patient based on the predicted future status. Benefits of predicting a future status of a patient condition are not limited to delivering therapy and administering medication. Clinicians may diagnose patient conditions, track patient conditions, or make other healthcare decisions based on a predicted future status of a patient condition.

According to the techniques of this disclosure, an insertable cardiac monitor (ICM) or other implantable medical device (IMD) may collect a cardiac signal such as an electrocardiogram (ECG) or an electrogram (EGM). Cardiac signals may include information that is relevant to monitoring a variety of patient conditions. For example, a cardiac signal may include fiducials that indicate events in the cardiac cycle. For example, a P-wave indicates an atrial depolarization, an R-wave indicates a ventricular depolarization, and a T-wave indicates a ventricular repolarization. The system may analyze the cardiac signal to identify these fiducials, or monitor one or more patient conditions based on identified fiducials and/or other information. For example, the system may classify the cardiac signal as indicating one or more patient conditions such as atrial fibrillation (AF).

The techniques of this disclosure may be implemented by systems including one or more IMDs that can autonomously and continuously collect biometric data and store biometric data while the one or more IMDs are implanted in a patient over months or years and perform numerous operations per second on the data to enable the systems herein to analyze the biometric data. Using techniques of this disclosure with an IMD may be advantageous when a physician cannot be continuously present with the patient to evaluate the physiological parameters and/or where performing the operations on the data described herein could not practically be performed in the mind of a physician.

In some examples, the techniques and systems of this disclosure may use one or more models, e.g., probability and/or machine learning models, to more accurately analyze biometric data to identify fiducials, monitor a patient condition, or predict a future status of a patient condition. In some examples, the model is trained with a set of training instances, where one or more of the training instances comprise data that indicate relationships between biometric data and likelihood values. Because the model is configured based on potentially thousands or millions of training instances, the model may reduce the amount of error in recurrence prediction performed by the system. Reducing errors using the techniques of this disclosure may provide one or more technical and clinical advantages, such as increasing the likelihood that the system will accurately provide information based analyzing the biometric data.

A machine learning model may provide a more accurate output when the model is trained using a larger set of training data as compared with when the model is trained using a smaller set of training data. This is because larger training datasets provide a greater number of training instances than smaller training datasets provide, meaning that models trained using larger training datasets generate outputs based on a greater amount of knowledge as compared with models trained using smaller training datasets. When a machine learning model is trained using supervised learning, the training data is labelled so that the model learns associations between data features and labels. This means that an accuracy of a machine learning model trained using supervised learning may be limited when an amount of available labeled training data is limited.

The techniques of this disclosure may provide one or more advantages. For example, a system may be configured to pretrain a machine learning model by processing unlabeled training data to acquire information corresponding to the unlabeled training data. Subsequently, the system may train the machine learning model using the processed unlabeled training data. One way that the system may process unlabeled training data is by predicting future portions of a training dataset based on past portions of the training dataset using autoregression, and comparing predicted training data against actual training data. In some examples, the system may process unlabeled training data by partitioning training datasets into a sequence overlapping data segments, the overlap attenuating issues arising at boundaries of data segments.

Pretraining a machine learning model by processing unlabeled training data may improve a performance of a machine learning model as compared with a system that does not pretrain a machine learning model by processing unlabeled training data, because the amount of available labeled training data may be limited. For example, partitioning training data into overlapping data segments, performing autoregression based on the overlapping data segments to generate a predicted dataset, and calculating a loss value based on the overlapping data segments and the predicted dataset may improve the machine learning model trained using the unlabeled training data as compared with systems that do not pretrain unlabeled training data. This is because partitioning training datasets into overlapping data segments may improve continuity of the predicted dataset generated using autoregression as compared with systems that do not partition training datasets into overlapping data segments.

In one example, a medical device system includes a medical device comprising one or more electrodes and configured to generate cardiac data based on a cardiac signal sensed via the one or more electrodes; and a memory configured to store: a machine learning model; a first plurality of training datasets, wherein each training dataset of the first plurality of training datasets not labeled with information corresponding to the dataset; and a second plurality of training datasets, wherein each training dataset of the second plurality of training datasets is labeled with information corresponding to the dataset. Additionally, the medical device system includes processing circuitry in communication with the memory, wherein the processing circuitry is configured to: receive, from the medical device, the cardiac data; and generate information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.

In another example, a method of operating a medical device system includes a medical device comprising one or more electrodes and configured to generate cardiac data based on a cardiac signal sensed via the one or more electrodes, the method comprising: receiving, by processing circuitry of the medical device system from the medical device, the cardiac data, wherein the processing circuitry is in communication with a memory of the medical device system. The memory is configured to store: a machine learning model; a first plurality of training datasets, wherein each training dataset of the first plurality of training datasets not labeled with information corresponding to the dataset; and a second plurality of training datasets, wherein each training dataset of the second plurality of training datasets is labeled with information corresponding to the dataset. Additionally, the method comprises generating, by the processing circuitry, information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.

In another example, a non-transitory computer-readable storage medium includes program instructions that, when executed by processing circuitry of a medical device system comprising one or more electrodes and configured to generate cardiac data based on a cardiac signal sensed via the one or more electrodes, cause the processing circuitry to: receive the cardiac data, wherein the processing circuitry is in communication with a memory of the medical device system. The memory is configured to store: a machine learning model; a first plurality of training datasets, wherein each training dataset of the first plurality of training datasets not labeled with information corresponding to the dataset; and a second plurality of training datasets, wherein each training dataset of the second plurality of training datasets is labeled with information corresponding to the dataset. Additionally, the instructions cause the processing circuitry to generate information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 is a block diagram illustrating an example medical device system including an implantable medical device (IMD) in conjunction with a patient 4, in accordance with one or more techniques of this disclosure.

FIG. 2A is a perspective drawing illustrating an IMD, which may be an example configuration of the IMD of FIG. 1 as an Insertable Cardiac Monitor (ICM), in accordance with one or more techniques of this disclosure.

FIG. 2B is a perspective drawing illustrating another IMD, which may be another example configuration of IMD from FIG. 1 as an ICM, in accordance with one or more techniques of this disclosure.

FIG. 3 is a block diagram illustrating an example configuration of the IMD of FIG. 1, in accordance with one or more techniques of this disclosure.

FIG. 4 is a block diagram illustrating an example configuration of a computing device, which may correspond to either (or both operating in coordination) of the computing devices of FIG. 1, in accordance with one or more techniques of this disclosure.

FIG. 5 is a block diagram illustrating an operating perspective of the health monitoring system (HMS) of FIG. 1, in accordance with one or more techniques of this disclosure.

FIG. 6 is a conceptual diagram illustrating an example machine learning model configured to output information corresponding to cardiac data, in accordance with one or more techniques of this disclosure.

FIG. 7 is a conceptual diagram illustrating a process for pretraining unlabeled training data, in accordance with one or more techniques of this disclosure.

FIG. 8 is a flow diagram illustrating an example method for using a machine learning model to generate information corresponding to cardiac data collected from a patient, in accordance with one or more techniques of this disclosure.

### DETAILED DESCRIPTION

This disclosure describes various systems, devices, and techniques for analyzing biometric data collected by a medical device to generate information relating to a health of a patient. In some examples, it may be possible to identify one or more patient conditions, such as atrial fibrillation (AF), atrioventricular (AV) block, or another cardiac arrhythmia, or predict a future occurrence of one or more patient conditions based on analyzing cardiac data such as an electrocardiogram (ECG) or an electrogram (EGM). A system may use a machine learning model to analyze cardiac data in order to generate information relating to a patient condition such as AF or AV block. In some cases, the system may train the machine learning model using a large set of training data that includes a combination of unlabeled training datasets and labeled training datasets.

In machine learning, “labeled” training data refers to training data that has information attached identifying one or more aspects of the data. For example, an EGM signal may include labels identifying P-waves, R-waves, and T-waves. Other example labels may include patient information such as age, sex, preexisting conditions, or any combination thereof. “Unlabeled” training data refers to training data that does not include information identifying aspects of the data. An unlabeled EGM training dataset may include EGM data including features such as P-waves, R-waves, and T-waves without any labels that identify such features or any other characteristics of the dataset.

A system may be configured to pretrain a machine learning model by processing unlabeled training datasets to acquire information corresponding to these unlabeled training datasets. By processing the unlabeled training datasets to acquire information, the system may increase the pool of available training data for training the machine learning model, and thus improve an accuracy of the machine learning model. For example, the system may use autoregression to predict future portions of an unlabeled training dataset based on past portions of an unlabeled training dataset. The system may determine an accuracy of these predictions and use this information to train the machine learning model. The system may, in some examples, also partition training datasets into overlapping data segments, add learned positional encoding to data segments, create a causal mask, or any combination thereof as part of pretraining the machine learning model.

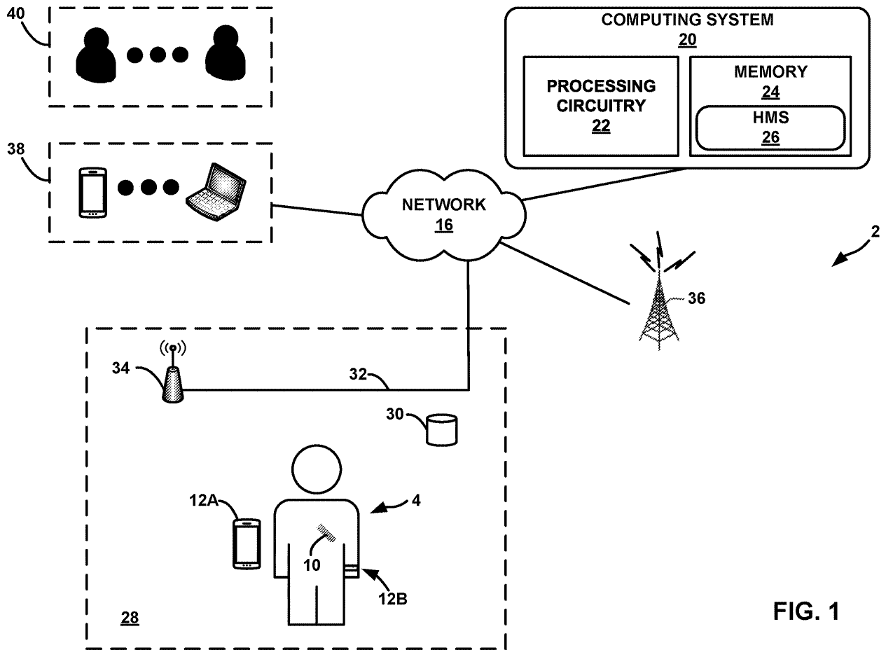

FIG. 1 is a block diagram illustrating an example medical device system 2 including an IMD 10 in conjunction with a patient 4, in accordance with one or more techniques of this disclosure. IMD 10 is configured for continuous, long-term monitoring of the heart of patient 4. For example, IMD 10 is configured to sense an ECG, an EGM, or other cardiac signal, identify fiducials in the cardiac signal, detect an arrythmia (e.g., AF, AV block) based on the cardiac signal, and store the cardiac signal and information corresponding to the cardiac signal to a memory of the IMD.

As examples, IMD 10 may be a pacemaker or implantable cardioverter-defibrillator, which may be coupled to intravascular or extravascular leads, or a pacemaker with a housing configured for implantation within the heart, which may be leadless. Some IMDs do not provide therapy, such as implantable patient monitors. IMD 10 may be such an IMD, e.g., the Reveal LINQ™ or LINQ II™ Insertable Cardiac Monitor (ICM), available from Medtronic, Inc., of Minneapolis, Minnesota, which may be inserted subcutaneously. Such IMDs may facilitate relatively longer-term and continuous monitoring of patients during normal daily activities, and may periodically transmit collected data to a remote patient monitoring system, such as the Medtronic Carelink™ Network.

IMD 10 may determine values of patient parameters or metrics, e.g., based on physiological signals sensed by the IMD or responsive therapies delivered by the IMD. The patient parameters may include, as examples, fluid level, heart rate, respiration rate, patient activity, temperature, heart sounds, oxygenation, and R-wave morphological characteristics. Other example patient metrics include coughing, speech, posture, tissue perfusion, hematocrit, thoracic impedance, subcutaneous impedance, intracardiac impedance, heart rate variability (HRV), weight, blood pressure, sleep apnea burden (which may be derived from respiration rate), ischemia burden, sleep duration, sleep quality, pre-ventricular contraction (PVC) burden, the occurrence, frequency or duration cardiac arrhythmias or other events, such as AF or AV block, and sensed cardiac intervals (e.g., Q-T intervals). Another example patient metric is the ventricular rate during AF. The concentration or levels of various substances, such as blood glucose, hematocrit, troponin and/or brain natriuretic peptide (BNP) levels, within the patient may also be used as one or more patient metrics.

In addition to IMD 10, system 2 includes one or more patient computing devices, e.g., patient computing devices 12A and 12B (collectively, “patient computing devices 12”). Patient computing device(s) 12 are configured for wireless communication with IMD 10. Computing device(s) 12 retrieve parameter data, e.g., AF episode data or AV block data, and other data from IMD 10. In some examples, computing device(s) 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing device(s) 12 may communicate with IMD 10 and each other according to the Bluetooth® or Bluetooth® Low Energy (BLE) protocols, as examples. In some examples, only one of computing device(s) 12, e.g., computing device 12A, is configured for communication with IMD 10, e.g., due to execution of software (e.g., part of a health monitoring application as described herein) enabling communication and interaction with an IMD.

In some examples, computing device(s) 12, e.g., wearable computing device 12B in the example illustrated by FIG. 1, may include electrodes and other sensors to sense physiological signals of patient 4, and may collect and store parameter data based on such signals. Computing device 12B may be incorporated into the apparel of patient 4, such as within clothing, shoes, eyeglasses, a watch or wristband, a hat, etc. In some examples, computing device 12B is a smartwatch or other accessory or peripheral for a smartphone computing device 12A.

One or more of computing device(s) 12 may be configured to communicate with a variety of other devices or systems via a network 16. For example, one or more of computing device(s) 12 may be configured to communicate with one or more computing systems, e.g., computing system 20, via network 16. Computing system 20 may be managed by a manufacturer of IMD 10 to, for example, provide cloud storage and analysis of collected data, maintenance and software services, or other networked functionality for their respective devices and users thereof. Computing system 20 may comprise, or may be implemented by, the Medtronic Carelink™ Network, in some examples.

As illustrated in FIG. 1, computing system 20 may include processing circuitry 22 and memory 24. Processing circuitry 22 may include fixed function circuitry and/or programmable processing circuitry. Processing circuitry 22 may include any one or more of a microprocessor, a controller, a digital signal processor (DSP), graphics processing unit (GPU), tensor processing unit (TPU), an application specific integrated circuit (ASIC), a field-programmable gate array (FPGA), or equivalent discrete or analog logic circuitry. In some examples, processing circuitry 22 may include multiple components, such as any combination of one or more microprocessors, one or more controllers, one or more DSPs, GPUs, TPUs, one or more ASICs, or one or more FPGAs, as well as other discrete or integrated logic circuitry, which may be physically located in one or more devices in one or more physical locations. Computing system 20 may be configured as a cloud computing system.

Processing circuitry 22 may be capable of processing instructions stored in memory 24. In some examples, memory 24 includes a computer-readable medium that includes instructions that, when executed by processing circuitry 22, cause computing system 20 and processing circuitry 22 to perform various functions attributed to them herein. In the example illustrated by FIG. 1, computing system 20 implements a health monitoring system (HMS) 26. As will be described in greater detail below, HMS 26 may generate information corresponding to one or more patient conditions of patient 4 based on physiological data collected by IMD 10. Memory 24 may include any volatile, non-volatile, magnetic, optical, or electrical media, such as a random-access memory (RAM), read-only memory (ROM), non-volatile RAM (NVRAM), electrically erasable programmable ROM (EEPROM), ferroelectric RAM (FRAM), dynamic random-access memory (DRAM), flash memory, or any other digital media.

Computing device(s) 12 may transmit data, including data retrieved from IMD 10, to computing system 20 via network 16. The data may include sensed data, e.g., values of physiological parameters measured by IMD 10 and, in some cases one or more of computing device(s) 12, and other physiological signals or data recorded by IMD 10 and/or computing device(s) 12. The data transmitted from computing device(s) 12 to computing system 20 may include cardiac data such as ECGs and/or EGMs.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMD 10 may be configured to generate diagnostic information of patient 4, such as information classifying one or more arrythmia episodes (e.g., AF episodes or AV block episodes). In some examples, IMD 10 may be configured to transmit such data to wireless access point 34 and/or computing device(s) 12. Wireless access points 34 and/or computing device(s) 12 may then communicate the retrieved data to computing systems 20 via network 16.

In some cases, computing system 20 may be configured to provide a secure storage site for data that has been collected from IMD 10 and/or computing device(s) 12. In some instances, computing system 20 may include a database that stores medical- and health-related data. For example, computing system 20 may include a cloud server or other remote server that stores data collected from IMDs 10 and/or computing device(s) 12. In some cases, computing system 20 may assemble data in web pages or other documents for viewing by trained professionals, such as clinicians 40, via clinician computing devices 38. One or more aspects of the example system described with reference to FIG. 1 may be implemented with general network technology and functionality, which may be similar to that provided by the Medtronic CareLink® Network.

In some examples, one or more of clinician computing devices 38 may be a tablet or other smart device located with a clinician, by which the clinician may program, receive alerts from, and/or interrogate IMD 10. For example, the clinician may access data collected by IMD 10 through a clinician computing device 38, such as when patient 4 is in between clinician visits, to check on a status of a medical condition. For example, computing system 20 may transmit data indicating one or more fiducials of a cardiac signal and/or an AF classification (e.g., determined by computing system 20, computing device 12, or other devices described herein) to clinician(s) 40 via computing device(s) 38.

As will be described herein, processing circuitry of one or more devices of medical device system 2 (e.g., of computing device 12 and/or computing system 20 implementing HMS 26) may be configured to determine information corresponding to cardiac data generated by IMD 10. In some examples, HMS 26 may include a machine learning model that is configured to generate an output based on receiving cardiac data generated by IMD 10 as an input. The machine learning model may, in some examples, generate an output that includes information corresponding to a health of patient 4. For example, the machine learning model may output classifications of one or more arrythmias (e.g., AF or AV block) present in cardiac data generated by IMD 10. In some cases, the machine learning model may output information indicating that patient 4 is likely to experience one or more patient conditions (e.g., AF or AV block) in the future. In some examples, the machine learning model may be stored by a memory of IMD 10, but this is not required. The machine learning model may be stored by a device separate from IMD 10.

In the example of FIG. 1, environment 28 includes one or more Internet of Things (IoT) devices, such as IoT device 30. IoT device 30 may include, as examples, so called “smart” speakers, cameras, televisions, lights, locks, thermostats, appliances, actuators, controllers, or any other smart home (or building) devices. In the example of FIG. 1, IoT device 30 is a smart speaker and/or controller, which may include a display. In some examples, IoT device 30 includes cameras or other sensors may activate those sensors to collect data regarding patient 4, e.g., for evaluation of the condition of patient 4.

Computing device(s) 12 may be configured to wirelessly communicate with IoT device 30 to cause IoT device 30 to take the actions described herein. In some examples, HMS 26 communicates with IoT device 30 via network 16 to cause IoT device 30 to take the actions described herein. In some examples, IMD 10 is configured to communicate wirelessly with IoT device 30, e.g., to communicate data to computing system 20 via network 16. In some examples, IoT device 30 may be configured to provide some or all of the functionality ascribed to computing device(s) 12 herein.

Environment 28 includes computing facilities, e.g., a local network 32, by which computing device(s) 12, IoT device 30, and other devices within environment 28 may communicate via network 16, e.g., with HMS 26. For example, environment 28 may be configured with wireless technology, such as the Institute of Electrical and Electronics Engineers (IEEE) 802.11 wireless networks, IEEE 802.15 ZigBee networks, an ultra-wideband protocol, near-field communication, or the like. Environment 28 may include one or more wireless access points, e.g., wireless access point 34 that provides support for wireless communications throughout environment 28. Additionally, or alternatively, e.g., when local network is unavailable, computing device(s) 12, IoT devices 30, and other devices within environment 28 may be configured to communicate with network 16, e.g., with HMS 26, via a cellular base station 36 and a cellular network.

In some examples, computing device(s) 12 and/or computing system 20 may implement one or more algorithms to determine information corresponding to patient 4 based on data received from IMD 10. In some examples, computing device(s) 12 and/or computing system 20 may have greater processing capacity than IMD 10, enabling more complex analysis of the data. In some examples, the computing device(s) 12 and/or HMS 26 may apply the data to a probability model, machine learning model or other artificial intelligence developed algorithm, e.g., to determine information corresponding to patient 4 described herein. Although described in the context of examples in which IMD 10 that senses patient cardiac activity may comprise an ICM, example systems including one or more implantable, wearable, or external devices of any type configured to sense physiological parameters of a patient may be configured to implement the techniques of this disclosure.

In some examples, IMD 10 includes one or more electrodes. IMD 10 may generate cardiac data based on a cardiac signal sensed via the one or more electrodes. In some examples, the cardiac data may be based on a cardiac electrical signal such as an ECG or an EGM that indicates one or more aspects of cardiac activity of patient 4. For example, the cardiac data may indicate one or more cardiac events such as atrial depolarizations (indicated by P-waves), ventricular depolarizations (indicated by R-waves), and ventricular repolarizations (indicated by T-waves). The cardiac data may represent a sequence of cardiac data points such that any given data point in the sequence of cardiac data points corresponds to a magnitude of the cardiac signal at a point in time. Based on the time at which cardiac events occur, the cardiac data may indicate one or more parameters such as heart rate and heart rate variability. IMD 10 may, in some examples, output the cardiac signal to computing system 20. Computing system 20 may, in some cases, store the cardiac signal to memory 24.

In some examples, memory 24 is configured to store a machine learning model. HMS 26 may be configured to execute the machine learning model using the cardiac signal received from IMD 10 as an input to generate an output. In some examples, memory 24 is configured to store a first plurality of training datasets, wherein each training dataset of the first plurality of training datasets is not labeled with information corresponding to the dataset. In some examples, memory 24 is configured to store a second plurality of training datasets, wherein each training dataset of the second plurality of training datasets is labeled with information corresponding to the dataset. In some examples, a training data set of the first plurality of training datasets that is not labeled with information corresponding to the dataset may include cardiac data that does not include information corresponding to one or more aspects of the cardiac data. For example, a training dataset that is not labeled might not have tags identifying fiducials such as P-waves, R-waves and T-waves. Additionally, or alternatively, a training dataset that is not labeled might not be associated with information corresponding to a patent associated with the training dataset, such as an age of the patient, a sex of the patient, and one or more patient conditions associated with the patient.

Processing circuitry 22 of computing system 20 may receive, from IMD 10, cardiac data from patient 4. In some examples, processing circuitry 22 may receive the cardiac data in real time or near real-time as a continuous stream of cardiac data. In some examples, processing circuitry 22 may receive the cardiac data as a set bloc of data representing a window of time prior to a current time. Processing circuitry 22 may generate information corresponding to cardiac data by executing the machine learning model of HMS 26. In some examples, machine learning model is pretrained without supervision using the first plurality of training data stored by memory 24. In some examples, the machine learning model is trained with supervision to identify the information corresponding to the cardiac data using the second plurality of training datasets stored by memory 24.

In some examples, to generate information corresponding to the cardiac data, the processing circuitry is configured to identify one or more fiducials of the cardiac data. Fiducials may represent areas of interest in a cardiac signal that correspond to areas of interest in a cardiac cycle. For example, a P-wave is a fiducial corresponding to an atrial depolarization, an R-wave is a fiducial corresponding to a ventricular depolarization, and a T-wave is a fiducial corresponding to a ventricular repolarization. This means that identifying fiducials in a cardiac signal may be beneficial for monitoring one or more patient conditions. For example, irregularities in P-waves present in a cardiac signal may indicate one or more atrial arrythmias, such as AF or AV block. As described herein, the term “fiducial” may refer to a point in a signal, a continuous set of points in a signal, or a discontinuous set of points in a signal.

To generate information corresponding to the cardiac data, processing circuitry 22 may be configured to determine whether the cardiac data indicates an arrythmia such as AF or AV block. Determining whether the cardiac data indicates an arrythmia may include identifying one or more fiducials of the cardiac signal, but this is not required. In any case, the machine learning model may process the cardiac data to identify whether the cardiac data indicates an arrythmia based on the cardiac signal and information available corresponding to the cardiac data.

Processing circuitry 22 may train the machine learning model based on the first plurality of training datasets and the second plurality of training datasets stored by the memory 24. In some examples, it may be beneficial for computing system 20 to acquire information corresponding to unlabeled training data so that computing system 20 is able to use unlabeled training data to train the machine learning model based on this acquired information. For example, processing circuitry 22 is configured to pretrain the machine learning model by, for each training dataset of the first plurality of training datasets not labeled with information corresponding to the data, performing autoregression to generate a predicted dataset corresponding to the training dataset. In some examples, performing autoregression to generate predicted datasets allows computing system 20 to identify information corresponding to unlabeled training datasets. For example, processing circuitry 22 may compare a predicted dataset generated using autoregression with a corresponding training dataset of the first plurality of training datasets. Based on the comparison, processing circuitry 22 may obtain information corresponding to the training dataset.

In some examples, to perform autoregression to generate a predicted dataset using a training dataset of the first plurality of training datasets, processing circuitry 22 is configured to predict, at each time point of a sequence of time points of the training dataset, a future portion of the training dataset based on a portion of the training dataset occurring before the time point. For example, processing circuitry 22 may use a first portion of a training dataset to predict a portion of data following the first portion of the training dataset. Processing circuitry 22 may use a first portion and a second portion of the training dataset to predict a portion of data following the second portion of the training dataset. Processing circuitry 22 may use a first portion, a second portion, and a third portion of the training dataset to predict a portion of data following the third portion of the training dataset, and so on.

Prior to performing autoregression, processing circuitry 22 may be configured to normalize each training dataset of the first plurality of training datasets by subtracting, from each data sample of the training dataset, a mean data sample value corresponding to the training dataset. Additionally, or alternatively, processing circuitry 22 may divide each data sample of the training dataset by a standard deviation data sample value corresponding to the training dataset. Normalizing training datasets may improve a manner in which processing circuitry 22 pretrains the machine learning model by centering training datasets around a baseline axis.

When processing circuitry 22 generates a predicted dataset corresponding to each training dataset of the first plurality of training datasets using autoregression, processing circuitry 22 may first partition each training dataset of the first plurality of training datasets into a plurality of data segments that form a sequence of overlapping data segments. That is, a training dataset may be partitioned into a sequence of overlapping data segments such that each data segment of the sequence may include one or more data samples that are also present in one or more other data segments of the sequence of overlapping data segments.

Processing circuitry 22 may compare predicted datasets with corresponding training datasets of the first plurality of training datasets in order to acquire information corresponding to unlabeled training datasets. To compare a predicted training dataset with a corresponding training dataset of the first plurality of training datasets, the processing circuitry is configured to calculate, for each data segment of the plurality of data segments of the training dataset, a loss value. In some examples, the loss value is based on a difference between a non-overlapping portion of the data segment of the training dataset and a corresponding portion of the predicted dataset.

In some examples, processing circuitry 22 is further configured to pretrain the machine learning model of HMS 26 by, for each training dataset of the first plurality of training datasets, applying positional encoding to each data segment of the plurality of data segments corresponding to the training dataset. In some examples, the positional encoding applied to each data segment of the plurality of data segments includes information corresponding to a position of the data segment within the sequence of overlapping data segments. This means that positional encoding inserts positional information into the training data that allows processing circuitry 22 to train the machine learning model based on relative a position of each data segment within the sequence of overlapping data segments.

In some examples, processing circuitry 22 is further configured to pretrain the machine learning model using the first plurality of training datasets. Processing circuitry 22 is configured to train the pretrained machine learning model using the second plurality of training data sets to generate the machine learning model. In this way, processing circuitry 22 trains the machine learning model using both the unlabeled first plurality of training datasets and the labeled second plurality of training datasets. Pretraining the machine learning model using the unlabeled first plurality of training datasets allows the processing circuitry 22 obtain information corresponding to the unlabeled first plurality of training datasets that is useful for training the machine learning model.

The techniques described herein are not limited to examples where IMD 10 comprises an ICM. In some examples, IMD 10 of FIG. 1 may include any kind of external or implantable medical device that is configured to collect a cardiac signal such as an ECG or an EGM. For example, IMD 10 may include an implantable cardioverter-defibrillator (ICD), a pacemaker, a cardiac resynchronization therapy pacemaker (CRT-P), a cardiac resynchronization therapy defibrillator (CRT-D), or an external cardiac signal sensing device.

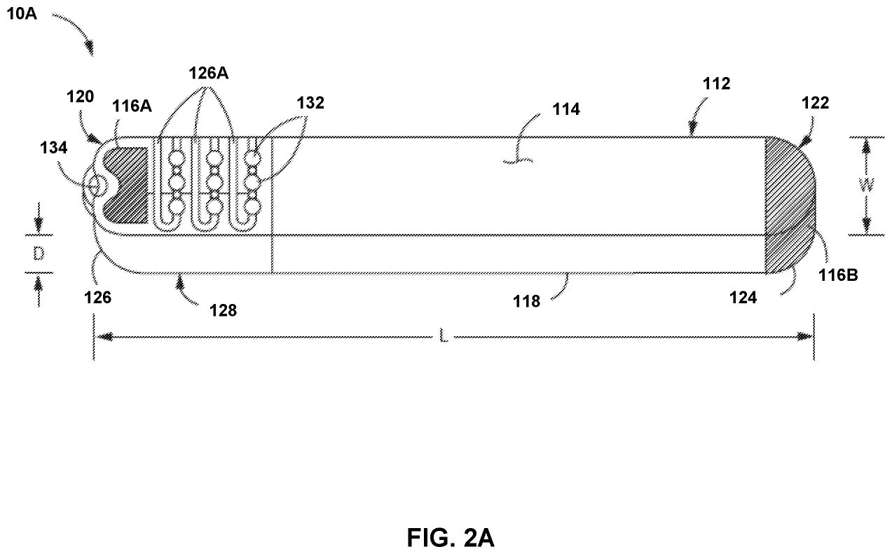

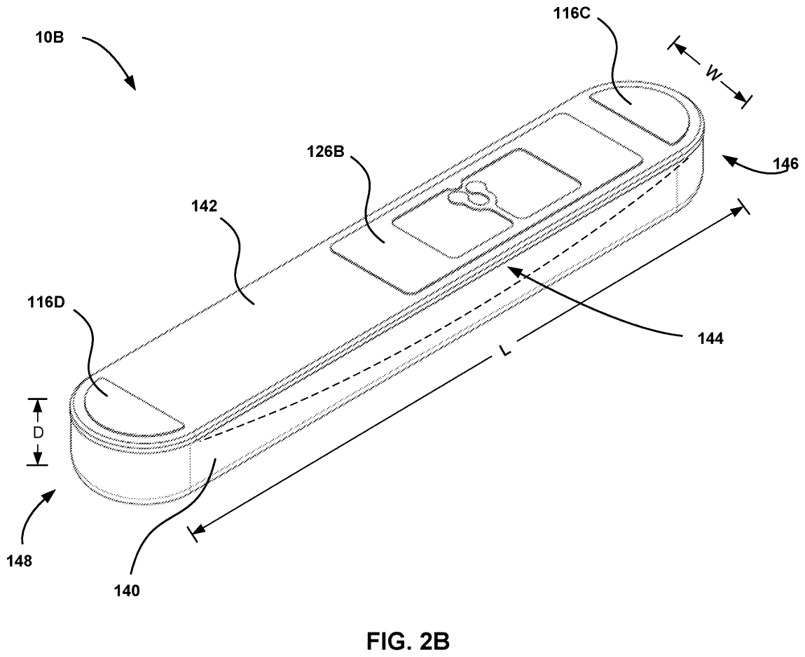

FIG. 2A is a perspective drawing illustrating an IMD 10A, which may be an example configuration of IMD 10 of FIG. 1 as an ICM, in accordance with one or more techniques of this disclosure. In the example shown in FIG. 2A, IMD 10A may be embodied as a monitoring device having housing 112, proximal electrode 116A and distal electrode 16B. Housing 112 may further comprise first major surface 114, second major surface 118, proximal end 120, and distal end 122. Housing 112 encloses electronic circuitry located inside the IMD 10A and protects the circuitry contained therein from body fluids. Housing 112 may be hermetically sealed and configured for subcutaneous implantation. Electrical feedthroughs provide electrical connection of electrodes 116A and 116B.

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A-in particular a width W greater than the depth D-is selected to allow IMD 10A to be inserted under the skin of the patient using a minimally invasive procedure and to remain in the desired orientation during insertion. For example, the device shown in FIG. 2A includes radial asymmetries (notably, the rectangular shape) along the longitudinal axis that maintains the device in the proper orientation following insertion. For example, the spacing between proximal electrode 116A and distal electrode 116B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 114 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 2A, once inserted within the patient, the first major surface 114 faces outward, toward the skin of the patient while the second major surface 118 is located opposite the first major surface 114. In addition, in the example shown in FIG. 2A, proximal end 120 and distal end 122 are rounded to reduce discomfort and irritation to surrounding tissue once inserted under the skin of the patient. IMD 10A, including instrument and method for inserting IMD 10A is described, for example, in U.S. Pat. No. 11,311,312, incorporated herein by reference in its entirety.

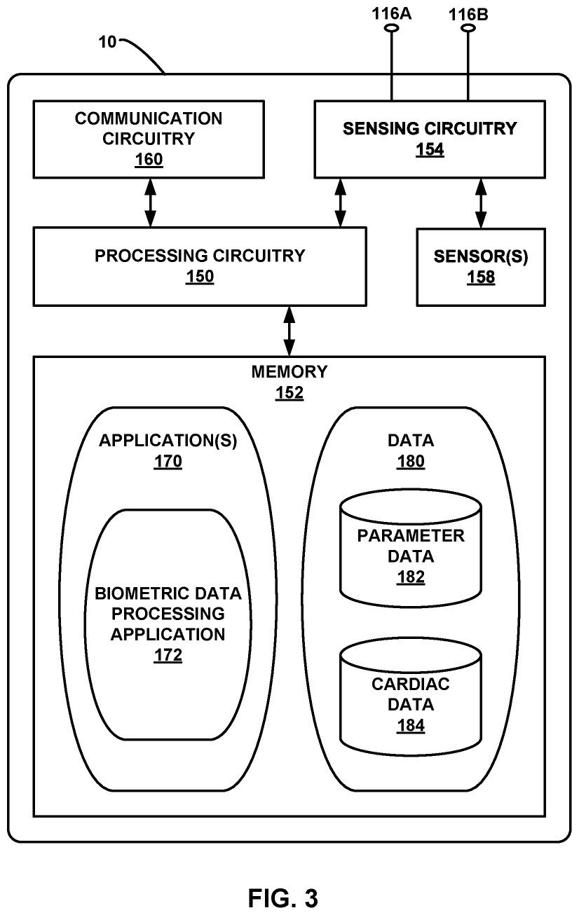

Proximal electrode 116A is at or proximate to proximal end 120, and distal electrode 116B is at or proximate to distal end 122. Proximal electrode 116A and distal electrode 116B are used to sense cardiac signals, e.g., ECG signals, and measure interstitial impedance thoracically outside the ribcage, which may be sub-muscularly or subcutaneously. ECG signals and impedance measurements may be stored in a memory of IMD 10A, and data may be transmitted via integrated antenna 126A to another device, which may be another implantable device or an external device, such as computing device 12. In some example, electrodes 116A and 116B may additionally or alternatively be used for sensing any bio-potential signal of interest, which may be, for example, an electrogram (EGM), electroencephalogram (EEG), electromyogram (EMG), or a nerve signal, from any implanted location. Housing 112 may house the circuitry of IMD 10 illustrated in FIG. 3.

In the example shown in FIG. 2A, proximal electrode 116A is at or in close proximity to the proximal end 120 and distal electrode 116B is at or in close proximity to distal end 122. In this example, distal electrode 116B is not limited to a flattened, outward facing surface, but may extend from first major surface 114 around rounded edges 124 and/or end surface 126 and onto the second major surface 118 so that the electrode 116B has a three-dimensional curved configuration. In some examples, electrode 116B is an uninsulated portion of a metallic, e.g., titanium, part of housing 112.

In the example shown in FIG. 2A, proximal electrode 116A is located on first major surface 114 and is substantially flat, and outward facing. However, in other examples proximal electrode 116A may utilize the three-dimensional curved configuration of distal electrode 116B, providing a three-dimensional proximal electrode (not shown in this example). Similarly, in other examples distal electrode 116B may utilize a substantially flat, outward facing electrode located on first major surface 114 similar to that shown with respect to proximal electrode 116A.

The various electrode configurations allow for configurations in which proximal electrode 116A and distal electrode 116B are located on both first major surface 114 and second major surface 118. In other configurations, such as that shown in FIG. 2A, only one of proximal electrode 116A and distal electrode 116B is located on both major surfaces 114 and 118, and in still other configurations both proximal electrode 116A and distal electrode 116B are located on one of the first major surface 114 or the second major surface 118 (e.g., proximal electrode 116A located on first major surface 114 while distal electrode 116B is located on second major surface 118). In another example, IMD 10A may include electrodes on both major surface 114 and 118 at or near the proximal and distal ends of the device, such that a total of four electrodes are included on IMD 10A. Electrodes 116A and 116B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 2A, proximal end 120 includes a header assembly 128 that includes one or more of proximal electrode 116A, integrated antenna 126A, anti-migration projections 132, and/or suture hole 134. Integrated antenna 126A is located on the same major surface (i.e., first major surface 114) as proximal electrode 116A and is also included as part of header assembly 128. Integrated antenna 126A allows IMD 10A to transmit and/or receive data. In other examples, integrated antenna 126A may be formed on the opposite major surface as proximal electrode 116A, or may be incorporated within the housing 112 of IMD 10A. In the example shown in FIG. 2A, anti-migration projections 132 are located adjacent to integrated antenna 126A and protrude away from first major surface 114 to prevent longitudinal movement of the device. In the example shown in FIG. 2A, anti-migration projections 132 include a plurality (e.g., nine) small bumps or protrusions extending away from first major surface 114. As discussed above, in other examples anti-migration projections 132 may be located on the opposite major surface as proximal electrode 116A and/or integrated antenna 126A. In addition, in the example shown in FIG. 2A, header assembly 128 includes suture hole 134, which provides another means of securing IMD 10A to the patient to prevent movement following insertion. In the example shown, suture hole 134 is located adjacent to proximal electrode 116A. In one example, header assembly 128 is a molded header assembly made from a polymeric or plastic material, which may be integrated or separable from the main portion of IMD 10A.

FIG. 2B is a perspective drawing illustrating another IMD 10B, which may be another example configuration of IMD 10 from FIG. 1 as an ICM, in accordance with one or more techniques of this disclosure. IMD 10B of FIG. 2B may be configured substantially similarly to IMD 10A of FIG. 2A, with differences between them discussed herein.

IMD 10B may include a leadless, subcutaneously implantable monitoring device, e.g., an ICM. IMD 10B includes housing having a base 140 and an insulative cover 142. Proximal electrode 116C and distal electrode 116D may be formed or placed on an outer surface of cover 142. Various circuitries and components of IMD 10B, e.g., described with respect to FIG. 3, may be formed or placed on an inner surface of cover 142, or within base 140. In some examples, a battery or other power source of IMD 10B may be included within base 140. In the illustrated example, antenna 126B is formed or placed on the outer surface of cover 142, but may be formed or placed on the inner surface in some examples. In some examples, insulative cover 142 may be positioned over an open base 140 such that base 140 and cover 142 enclose the circuitries and other components and protect them from fluids such as body fluids. The housing including base 140 and insulative cover 142 may be hermetically sealed and configured for subcutaneous implantation.

Circuitries and components may be formed on the inner side of insulative cover 142, such as by using flip-chip technology. Insulative cover 142 may be flipped onto a base 140. When flipped and placed onto base 140, the components of IMD 10B formed on the inner side of insulative cover 142 may be positioned in a gap 144 defined by base 140. Electrodes 116C and 116D and antenna 126B may be electrically connected to circuitry formed on the inner side of insulative cover 142 through one or more vias (not shown) formed through insulative cover 942. Insulative cover 142 may be formed of sapphire (i.e., corundum), glass, parylene, and/or any other suitable insulating material. Base 140 may be formed from titanium or any other suitable material (e.g., a biocompatible material). Electrodes 116C and 116D may be formed from any of stainless steel, titanium, platinum, iridium, or alloys thereof. In addition, electrodes 116C and 116D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between proximal electrode 116C and distal electrode 116D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within the patient, outer surface of cover 142 faces outward, toward the skin of the patient. In addition, as shown in FIG. 2B, proximal end 146 and distal end 148 are rounded to reduce discomfort and irritation to surrounding tissue once inserted under the skin of the patient. In addition, edges of IMD 10B may be rounded.

FIG. 3 is a block diagram illustrating an example configuration of IMD 10 of FIG. 1, in accordance with one or more techniques of this disclosure. As shown in FIG. 3, IMD 10 includes processing circuitry 150, memory 152, sensing circuitry 154 coupled to electrodes 116A and 116B (hereinafter, “electrodes 116”) and one or more sensor(s) 158, and communication circuitry 160.

Processing circuitry 150 may include fixed function circuitry and/or programmable processing circuitry. Processing circuitry 150 may include any one or more of a microprocessor, a controller, a GPU, a TPU, a DSP, an ASIC, a FPGA, or equivalent discrete or analog logic circuitry. In some examples, processing circuitry 150 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 150 herein may be embodied as software, firmware, hardware, or any combination thereof. In some examples, memory 152 includes computer-readable instructions that, when executed by processing circuitry 150, cause IMD 10 and processing circuitry 150 to perform various functions attributed herein to IMD 10 and processing circuitry 150. Memory 152 may include any volatile, non-volatile, magnetic, optical, or electrical media, such as a RAM, ROM, NVRAM, EEPROM, flash memory, or any other digital media.

Sensing circuitry 154 may sense an ECG and measure impedance, e.g., of tissue proximate to IMD 10, via electrodes 116. The measured impedance may vary based on respiration, cardiac pulse or flow, and a degree of perfusion or edema. Processing circuitry 150 may determine patient metrics relating to respiration, fluid retention, cardiac pulse or flow, perfusion, and/or edema based on the measured impedance. In some examples, processing circuitry 150 may identify features of the sensed ECG, such as heart rate, heart rate variability, T-wave alternans, intra-beat intervals (e.g., QT intervals), and/or ECG morphologic features, to detect an episode of cardiac arrhythmia of patient 4.

In some examples, IMD 10 includes one or more sensors 158, such as one or more accelerometers, gyroscopes, microphones, optical sensors, temperature sensors, pressure sensors, and/or chemical sensors. In some examples, sensing circuitry 154 may include one or more filters and amplifiers for filtering and amplifying signals received from one or more of electrodes 116 and/or sensors 158. In some examples, sensing circuitry 154 and/or processing circuitry 150 may include a rectifier, filter and/or amplifier, a sense amplifier, comparator, and/or analog-to-digital converter. Processing circuitry 150 may determine physiological parameter data 182, e.g., values of physiological parameters of patient 4, based on signals from sensors 158, which may be stored as data 180 in memory 152. Patient parameters determined from signals from sensors 158 may include intravascular fluid level, interstitial fluid level, oxygen saturation, glucose level, stress hormone level, heart sounds, body motion, activity intensity, sleep duration, sleep quality, body posture, or blood pressure.

In addition to data 180, memory 152 may store applications 170 executable by processing circuitry 150. Applications 170 may include a biometric data processing application 172. Processing circuitry 150 may execute biometric data processing application 172 to process parameter data 182 and cardiac data 184. In some examples, biometric data processing application 172 may identify information corresponding to parameter data 182 and/or cardiac data 184. For example, biometric data processing application 172 may identify a heart rate of patient 4 based on cardiac data 184 by identifying a rate at which R-waves occur in cardiac data 184.

Data 180 may include cardiac data 184 sensed by IMD 10 via electrodes 116. In some examples, cardiac data 184 may represent a cardiac signal such as an ECG or an EGM that indicates cardiac activity of patient 4. In some examples, cardiac data 184 may indicate cardiac activity of patient 4 over a long period of time (e.g., weeks or months) continuously collected via electrodes 116 while IMD 10 is implanted underneath the skin of patient 4. In some examples, cardiac data 184 may include a plurality of sets of cardiac data each collected via electrodes 116 when IMD 10 is implanted underneath the skin of patient 4. Cardiac data 184 may include a data indicating a cardiac signal, heart rate information, R-R interval information, ECG morphological information, or other information.

Processing circuitry 150 may communicate parameter data 182 and cardiac data 184 to one or more other computing devices, e.g., computing device(s) 12 and/or computing system 20, using communication circuitry 160. Communication circuitry 160 may include any suitable hardware, firmware, software or any combination thereof for wirelessly communicating with another device. Communication circuitry 160 may be configured to transmit and/or receive signals via inductive coupling, electromagnetic coupling, Near Field Communication (NFC), Radio Frequency (RF) communication, Bluetooth®, Wi-Fi, or other proprietary or non-proprietary wireless communication schemes.

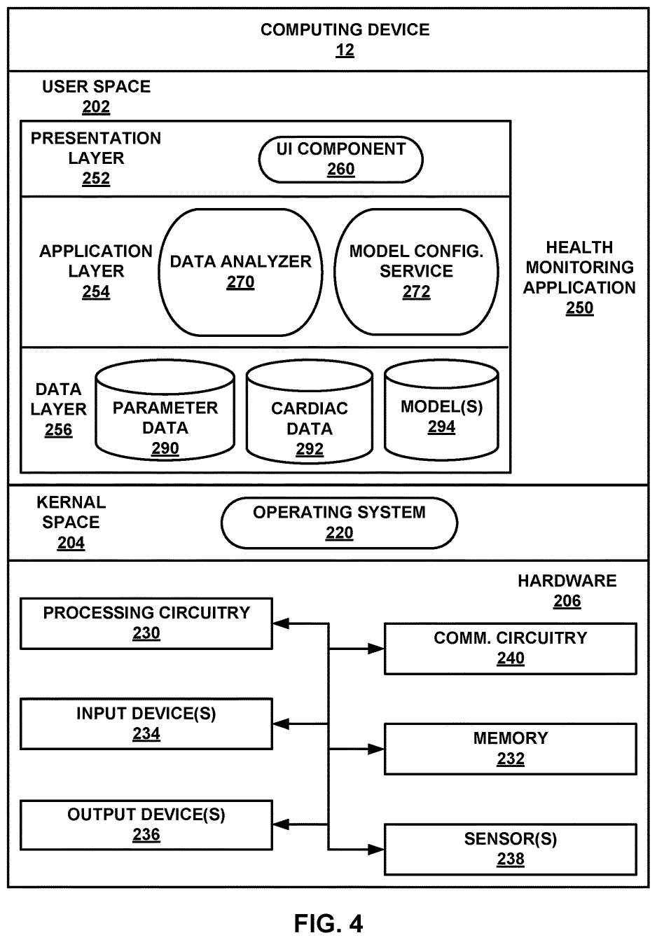

FIG. 4 is a block diagram illustrating an example configuration of a computing device 12, which may correspond to either (or both operating in coordination) of computing devices 12A and 12B, in accordance with one or more techniques of this disclosure. In some examples, computing device 12 takes the form of a smartphone, a laptop, a tablet computer, a personal digital assistant (PDA), a smartwatch or other wearable computing device. In some examples, IoT devices 30 and/or computing devices 38 and 42 may be configured similarly to the configuration of computing device 12 illustrated in FIG. 4.

As shown in the example of FIG. 4, computing device 12 may be logically divided into user space 202, kernel space 204, and hardware 206. Hardware 206 may include one or more hardware components that provide an operating environment for components executing in user space 202 and kernel space 204. User space 202 and kernel space 204 may represent different sections or segmentations of memory, where kernel space 204 provides higher privileges to processes and threads than user space 202. For instance, kernel space 204 may include operating system 220, which operates with higher privileges than components executing in user space 202.

As shown in FIG. 4, hardware 206 includes processing circuitry 230, memory 232, one or more input device(s) 234, one or more output device(s) 236, one or more sensor(s) 238, and communication circuitry 240. Although shown in FIG. 4 as a stand-alone device for purposes of example, computing device 12 may be any component or system that includes processing circuitry or other suitable computing environment for executing software instructions and, for example, need not necessarily include one or more elements shown in FIG. 4.

Processing circuitry 230 is configured to implement functionality and/or process instructions for execution within computing device 12. For example, processing circuitry 230 may be configured to receive and process instructions stored in memory 232 that provide functionality of components included in kernel space 204 and user space 202 to perform one or more operations in accordance with techniques of this disclosure. Examples of processing circuitry 230 may include, any one or more microprocessors, controllers, GPUs, TPUs, DSPs, ASICs, FPGAs, or equivalent discrete or integrated logic circuitry.

Memory 232 may be configured to store information within computing device 12, for processing during operation of computing device 12. Memory 232, in some examples, is described as a computer-readable storage medium. In some examples, memory 232 includes a temporary memory or a volatile memory. Examples of volatile memories include RAM, DRAM, static random access memory (SRAM), and other forms of volatile memories known in the art. Memory 232, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or EEPROM memories. In some examples, memory 232 includes cloud-associated storage.

One or more input device(s) 234 of computing device 12 may receive input, e.g., from patient 4, clinician 40, or another user. Examples of input are tactile, audio, kinetic, and optical input. Input device(s) 234 may include, as examples, a mouse, keyboard, voice responsive system, camera, buttons, control pad, microphone, presence-sensitive or touch-sensitive component (e.g., screen), or any other device for detecting input from a user or a machine.

One or more output device(s) 236 of computing device 12 may generate output, e.g., to patient 4 or another user. Examples of output are tactile, haptic, audio, and visual output. Output device(s) 236 of computing device 12 may include a presence-sensitive screen, sound card, video graphics adapter card, speaker, cathode ray tube monitor, liquid crystal display (LCD), light emitting diodes (LEDs), or any type of device for generating tactile, audio, and/or visual output.

One or more sensor(s) 238 of computing device 12 may sense physiological parameters or signals of patient 4. Sensor(s) 238 may include electrodes, accelerometers (e.g., 3-axis accelerometers), an optical sensor, impedance sensors, temperature sensors, pressure sensors, heart sound sensors (e.g., microphones or accelerometers), and other sensors, and sensing circuitry (e.g., including an analog-to-digital converter (ADC)), similar to those described above with respect to IMDs 10 and FIG. 3.

Communication circuitry 240 of computing device 12 may communicate with other devices by transmitting and receiving data. Communication circuitry 240 may receive data from IMD 10, such as patients metrics and/or higher resolution diagnostic information, from communication circuitry in IMD 10. Communication circuitry 240 may include a network interface card, such as an Ethernet card, an optical transceiver, a radio frequency transceiver, or any other type of device that can send and receive information. For example, communication circuitry 160 may include a radio transceiver configured for communication according to standards or protocols, such as 3G, 4G, 5G, Wi-Fi (e.g., 802.11 or 802.15 ZigBee), Bluetooth®, or BLE.

As shown in FIG. 4, health monitoring application 250 executes in user space 202 of computing device 12. Health monitoring application 250 may be logically divided into presentation layer 252, application layer 254, and data layer 256. Presentation layer 252 may include a user interface (UI) component 260, which generates and renders user interfaces of health monitoring application 250.

Data layer 256 may include parameter data 290 and cardiac data 292, which may be received from IMD 10 via communication circuitry 240, and stored in memory 232 by processing circuitry 230. Application layer 254 may include, but is not limited to, data analyzer 270, and model configuration service 272. Data analyzer 270 may be configured to process parameter data 290 and/or cardiac data 292 generated by IMD 10 to generate information corresponding to one or more patient conditions of patient 4. Data analyzer 270 may determine the information corresponding to patient 4 based on application of parameter data 290 and/or cardiac data 292 as inputs one or more model(s) 294, which may include one or more probability models, machine learning models, algorithms, decision trees, and/or thresholds. In examples in which model(s) 294 include one or more machine learning models, data analyzer 270 may apply feature vectors derived from the data to the model(s) 294.

Model configuration service 272 may be configured to modify model(s) 294 based on feedback indicating whether determinations were accurate or updated parameters received, e.g., from HMS 26. In some examples, model configuration service 272 may utilize the data sets from patient 4 for supervised machine learning to further train models included as part of model(s) 294. Model configuration service 272, or another component executed by processing circuitry of system 2, may select a configuration of model(s) 294 based on etiological data for patient. In some examples, different model(s) 294 tailored to different cohorts of patients may be available for selection for patient 4 based on such etiological data.

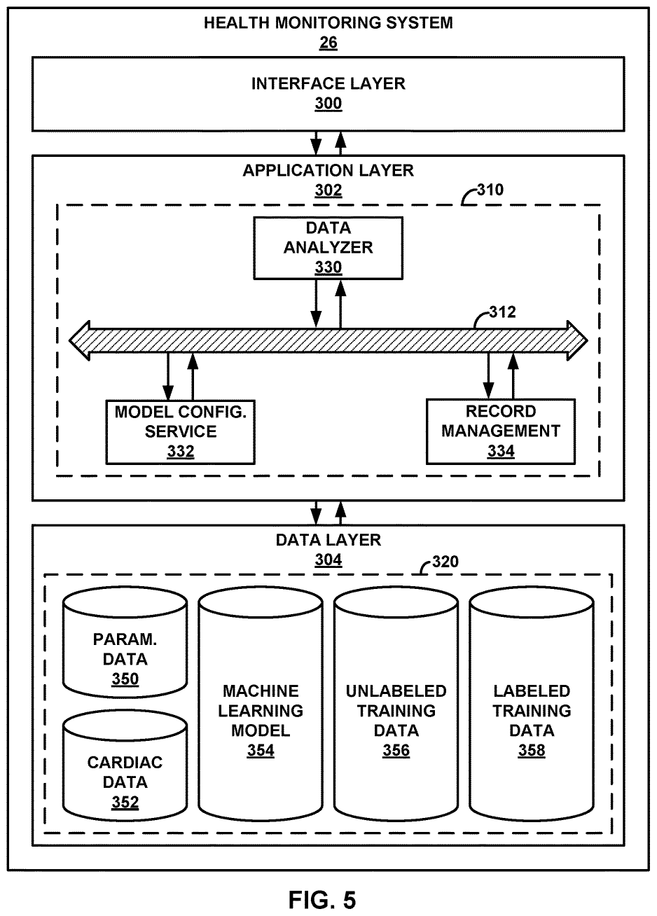

FIG. 5 is a block diagram illustrating an operating perspective of HMS 26, in accordance with one or more techniques of this disclosure. HMS 26 may be implemented in a computing system 20, which may include hardware components such as processing circuitry 22, memory 24, and communication circuitry, embodied in one or more physical devices. FIG. 5 provides an operating perspective of HMS 26 when hosted as a cloud-based platform. In the example of FIG. 5, components of HMS 26 are arranged according to multiple logical layers that implement the techniques of this disclosure. Each layer may be implemented by one or more modules comprised of hardware, software, or a combination of hardware and software.

Computing devices, such as computing device(s) 12, IoT devices 30, and computing devices 38 operate as clients that communicate with HMS 26 via interface layer 300. The computing devices typically execute client software applications, such as desktop application, mobile application, and web applications. Interface layer 300 represents a set of application programming interfaces (API) or protocol interfaces presented and supported by HMS 26 for the client software applications. Interface layer 300 may be implemented with one or more web servers.

As shown in FIG. 5, HMS 26 also includes an application layer 302 that represents a collection of services 310 for implementing the functionality ascribed to HMS 26 herein. Application layer 302 receives information from client applications, e.g., data from a computing device 12 or IoT device 30 (some or all of which may have been retrieved from IMD 10), and further processes the information according to one or more of the services 310 to respond to the information. Application layer 302 may be implemented as one or more discrete software services 310 executing on one or more application servers, e.g., physical or virtual machines. That is, the application servers provide runtime environments for execution of services 310. In some examples, the functionality interface layer 300 as described above and the functionality of application layer 302 may be implemented at the same server. Services 310 may communicate via a logical service bus 312. Service bus 312 generally represents a logical interconnection or set of interfaces that allows different services 310 to send messages to other services, such as by a publish/subscription communication model.

Data layer 304 of HMS 26 provides persistence for information in HMS 26 using one or more data repositories 320. A data repository 320, generally, may be any data structure or software that stores and/or manages data. Examples of data repositories 320 include but are not limited to relational databases, multi-dimensional databases, maps, and hash tables, to name only a few examples.

As shown in FIG. 5, each of services 330-334 is implemented in a modular form within HMS 26. Although shown as separate modules for each service, in some examples the functionality of two or more services may be combined into a single module or component. Each of services 330-334 may be implemented in software, hardware, or a combination of hardware and software. Moreover, services 330-334 may be implemented as standalone devices, separate virtual machines or containers, processes, threads or software instructions generally for execution on one or more physical processors. Record management service 334 may store received patient data as parameter data 350 and cardiac data 352.

Data analyzer 330 may determine information corresponding to cardiac activity of patient 4 based on cardiac data 352, and in some cases other parameter data 350, generated by IMD 10. In some examples, data analyzer 330 may identify one or more fiducials in cardiac data 352 received from IMD 10. In some examples, data analyzer 330 may determine whether cardiac data 352 indicates an arrythmia such as AF or AV block. Data analyzer 330 may determine the information corresponding to patient 4 based on application of parameter data 350 and/or cardiac data 352 as inputs to machine learning model 354. In some examples, data analyzer 330 may apply feature vectors derived from the data to machine learning model 354.

Machine learning model 354 may be developed by model configuration service 332. Example machine learning techniques that may be employed to generate machine learning model 354 include various learning styles, such as supervised learning, unsupervised learning, and semi-supervised learning. Example types of algorithms include Bayesian algorithms, Markov models, Hawkes processes, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, self-attention models, Convolutional Neural Networks (CNNs), Long Short Term Networks (LSTMs), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, model configuration service 332 may be configured to train machine learning model 354 using unlabeled training data 356 and labeled training data 358. In some examples, to train machine learning model 354, it may be beneficial for model configuration service to use information corresponding to training data in order to associate patterns and aspects of training data with known characteristics of the training data. For example, when model configuration service 332 is training machine learning model 354 to identify fiducials in cardiac signals such as ECG signals and EGM signals, it may be beneficial for the training data to include a plurality of cardiac data samples having labels that identify fiducials (e.g., labels identifying P-eaves, R-waves, T-waves and other fiducials). Additionally, or alternatively, when model configuration service 332 is training machine learning model 354 to identify arrythmias such as AF or AV block in cardiac data, it may be beneficial for the training data to include one or more labels indicating portions of the training data that indicate arrythmia.

In some examples, unlabeled training data 356 includes a plurality of training datasets that each represent a cardiac data without labels attached that provide information corresponding to the data. For example, each training dataset of unlabeled training data 356 may include a sample of cardiac data without any information attached identifying fiducials or portions corresponding to an arrythmia such as AF, AV block, or other kinds of arrythmia. On the other hand, labeled training data 358 may include a plurality of training datasets that each include one or more labels providing information corresponding to the dataset. For example, a training dataset of labeled training data 358 may include one or more labels indicating fiducials, one or more labels identifying portions of the dataset corresponding to AF episodes or other kinds of arrythmia, one or more labels providing information corresponding to the patient associated with the training dataset (e.g., age, sex, patient conditions), or any combination thereof.

In some examples, labeled training data 358 may be more useful for training machine learning model 354 than unlabeled training data 356 is for training machine learning model 354, because model configuration service 332 may be configured to learn one or more associations between labels attached to labeled training data 358 and characteristics of the training data associated with these labels. But in some cases, an amount of labeled training data 358 may be limited such that it is beneficial to use unlabeled training data 356 in addition to labeled training data 358 to improve an accuracy of machine learning model 354 as compared with systems that do not use both unlabeled training data 356 and labeled training data 358 to train a machine learning model.

When using unlabeled training data 356 to train machine learning model 354, model configuration service 332 may pretrain machine learning model 354 processing unlabeled training data 356 to identify information corresponding to unlabeled training data 356. That is, since each unlabeled training dataset of unlabeled training data 356 does not include labels providing information corresponding to the dataset, model configuration service 332 may process each training dataset of unlabeled training data 356 to discover information that is useful for training machine learning model 354.

In some examples, machine learning models such as CNNs and attention-based networks such as transformers are configured to segment and classify time series signals such as ECG signals and/or EGM signals. In some cases, an amount of labeled training datasets for training these models may be limited. That is, even when an accuracy of a machine learning model might be improved if a pool of labeled training data is larger as compared with smaller, an accuracy of the machine learning model might be limited when the pool of labeled training data is limited. This means that it may be beneficial to pretrain machine learning models for analyzing time series data by processing unlabeled training data to identify information that is useful for training a model using the unlabeled training data. Pretraining machine learning model by processing unlabeled training data to augment labeled training data may improve an accuracy of the model as compared with training the model using a smaller set of labeled training data without using unlabeled training data. In some examples, model configuration service 332 may process unlabeled training data 356 in an autoregressive fashion to predict future overlapping portions of cardiac data, and thereby learn generalizable features without needing labels that are present in labeled training data 358. Pretraining machine learning model 354 may lead to performance improvements in both segmentation and classification. By pretraining machine learning model 354, health monitoring system 26 may decrease a need for labels to achieve equivalent performance or improve performance given a fixed amount of labeled training data 358 as compared with systems that do not process unlabeled training data to pretrain machine learning models.

Once machine learning model 354 is trained using unlabeled training data 356 and labeled training data 358, data analyzer 330 may execute machine learning model 354 to perform arrythmia classification (e.g., AF classification or other arrythmia classification), perform segmentation to locate fiducials (e.g., P-waves, QRS complexes, and T-waves), or any combination thereof. For example, data analyzer 330 may execute machine learning model 354 to process cardiac data 352 to generate an output, the output including one or more arrythmia classifications and/or one or more identified fiducials. Data analyzer 330 may save the output from the machine learning model 354 to memory 24 of computing system 20.

Machine learning model 354 may, in some cases, comprise a stack of self-attention layers including decoders. Machine learning model 354 may include any number of heads, anu size of hidden layers, any number of layers, and any length of patches. In some examples, when machine learning model 354 is larger, machine learning model 354 performs better as compared with systems that use smaller machine learning models.

Self-supervised learning may be useful to training machine learning models for as natural language processing, computer vision (CV), and time series classification. By leveraging knowledge of a structure of underlying data, health monitoring system 26 may develop a pretraining task that encourages machine learning model 354 to learn general features about the underlying data distribution. These learned representations may be valuable for executing the machine learning model 354 to perform downstream tasks such as classification (e.g., AF classification or other arrythmia classification), semantic segmentation (e.g., identifying fiducials), and generation. When data is ordered, such as in natural language processing (NLP) or time series datasets, predicting future data based on past data using autoregression may be an effective pretraining method. By pretraining machine learning model 354, health monitoring system 26 may cause machine learning model 354 to develop a rich understanding of trends, patterns, and morphology of time series data to accurately predict future data.

In some examples, contrastive learning may be an effective pretraining objective for improving performance in downstream time series tasks. Health monitoring system 26 may develop a contrastive task in a supervised manner leveraging known or pseudo labels or in an unsupervised manner by leveraging an underlying structure of the data. For example, a system may leverage different augmentations for unlabeled data to develop similar and dissimilar signals for a contrastive loss. In some examples, a system first uses a supervised model to generate pseudo labels for an unlabeled dataset, and then constructs a contrastive loss using those pseudo labels. Both approaches may demonstrate performance improvements relative to a supervised baseline.

Pretraining cardiac signals (e.g., ECG signals and EGM signals) using future prediction may be a productive way to improve machine learning model 354. For example, health monitoring system 26 may adapt contrastive predictive encoding to the ECG domain and leverage a convolutional network to embed frames (patches) of the signal. Then, health monitoring system 26 may use a small transformer network to attend to the sequence of patches, the model trained in an autoregressive task.

Another pretraining approach includes leveraging data from other domains. For example, a classification model pretrained using a database may outperform a randomly initialized convolutional model trained for classification. Health monitoring system 26 may transform a one-dimensional time series into a two-dimensional representation or transform a two-dimensional representation into a one-dimensional time series. Health monitoring system 26 may plot spectrograms or other features, fold a signal into two dimensions, and take a two-dimensional plot of raw data. Large datasets may exist for pretraining models using both labeled and unlabeled training data. Long short-term memory (LSTM) models may be configured to handle the variable length nature of a sequence for segmentation and classification. In addition, or alternatively to using LSTM models, health monitoring system 26 may use graph architectures. Additionally, or alternatively, health monitoring system 26 may use convolutional models.

In addition to deep learning methods, health monitoring system 26 may use classical approaches for time series classification and segmentation. In some examples, heath monitoring system 26 may use the Pan-Tompkins algorithm for segmenting a cardiac signal into R-wave peak locations, variants of the Pan-Tompkins algorithm. In some cases, it may be beneficial to use deep learning models to perform segmentation.

In some examples, health monitoring system 26 may use autoregression (e.g., future prediction) as a mechanism for pretraining machine learning model 354. Pretraining machine learning model 354 by processing unlabeled training data 356 using autoregression may produce a more accurate machine learning model 354 as compared with an accuracy of models trained using masked-token prediction models such as bidirectional encoder representations from transformers (BERT) and contrastive models.

Prior to using autoregression to process unlabeled training data 356, model configuration service 332 may normalize each training dataset of unlabeled training data 356. One way that model configuration service 332 may normalize a training dataset is to subtract the global mean of the training dataset from each data sample of the training dataset and divide each data sample of the training dataset by the global standard deviation of the training dataset. Normalizing training data may be beneficial for finetuning downstream tasks such as segmentation (e.g., identifying fiducials). Another way that model configuration service 332 may normalize a training dataset is to take a log or take a cube root of each data sample of the training dataset.

In some examples, model configuration service 332 may perform data augmentation on unlabeled training data 356 to improve generalization of machine learning model 354. For example, model configuration service 332 may randomly permute small chunks of unlabeled training data 356 to perform augmentation for training machine learning model 354 to perform classification tasks. By augmenting unlabeled training data 356, model configuration service 332 may cause machine learning model 354 to develop features in medium to short time intervals relevant to tasks such as arrythmia classification (e.g., AF classification or other arrythmia classification). Furthermore, model configuration service 332 may use random crops to improve a performance of machine learning model 354.

Health monitoring system 26 may use temporal predictions for self-supervision. For example, model configuration service 332 may use autoregression to predict future portions of an unlabeled training dataset based on past portions of the unlabeled training dataset. In some examples, health monitoring system 29 may use a data patching (e.g., windowing) method to include overlap at the temporally near end of each patch of a predicted dataset generated using autoregression. For example, processing circuitry 22 may control health monitoring system 26 to partition a predicted dataset generated using autoregression based on an unlabeled training dataset of unlabeled training data 356 into a plurality of data segments. In some examples, each data segment of the plurality of data segments includes a set of data samples that are also present in one or more other data segments of the plurality of data segments such that the plurality of data segments of the predicted dataset forms a sequence of overlapping data segments.

In some examples, processing circuitry 22 may control model configuration service 332 of health monitoring system 26 to generate a predicted dataset based on each training dataset present in unlabeled training data 356. Processing circuitry 22 may, in some cases, control model configuration service 332 of health monitoring system 26 to each training dataset of unlabeled training data 356 into a sequence of overlapping data segments prior to generating a predicted dataset. The term “data segment” may, in some cases, be referred to herein as a “patch.” In some examples, model configuration service 332 may generate a predicted dataset based on an unlabeled training dataset. Model configuration service 332 may partition the training dataset into a sequence of data segments (e.g., a sequence of patches) each having a constant length.

In some examples where model configuration service 332 trains machine learning model 354 for segmentation tasks by adding overlap to the sequence of data segments improves machine learning model 354 as compared with systems that partition datasets without adding overlap. In one example, model configuration service 332 may partition a training dataset such that each data segment of the sequence of overlapping data segments includes 32 data samples, with 10 samples overlapping with one or more other data segments. The techniques of this disclosure are not limited to data segments having 32 data samples and 10 overlapping data samples. Data segments may include any number of data samples and any number of overlapping data samples. In some examples, using overlapping data segments may reduce edge effects on the borders of data segments when model configuration service 332 trains machine learning model 354 for segmentation tasks. For example, when no overlap is introduced, a finetuned model may predict a consistent U-shape with the data segment edges being higher probability despite data segment boundaries being uncorrelated with the input data. Model configuration service 332 may introduce overlap in the sequence of data segments to reduce these boundary issues.

In some examples, model configuration service 332 may introduce learned positional encoding to each sequence of overlapping data segments partitioned from a training dataset. In some examples, positional encoding applied to each data segment of the sequence of overlapping data segments includes information corresponding to a position of the data segment within the sequence of overlapping data segments. Consequently, positional encoding represents useful information for training machine learning model 354.

When model configuration service 332 generates a predicted dataset corresponding to each training dataset of unlabeled training data 356, model configuration service 332 may calculate one or more loss values corresponding to each training dataset of unlabeled training data 356 that reflect an accuracy at which health monitoring system 26 predicted future data points. For each training dataset of unlabeled training data 356, model configuration service 332 may calculate a set of loss values. Each loss value of the set of loss values may correspond to a data segment of the sequence of overlapping data segments of the training dataset. To calculate the set of loss values, model configuration service 332 may calculate a mean squared error loss corresponding to each non-overlapping portion of the set of overlapping data segments. The mean-squared error loss values may cause machine learning model 354 to develop predictive capability for long and broad features such as T-waves. Machine learning model 354 is not limited to predicting long and broad features such as T-waves. Machine learning model 354 may predict short and/or sharp features such as QRS complexes. In some examples, model configuration service 332 may calculate a single loss value for a training dataset based on a set of loss values each corresponding to a data segment of a sequence of data segments. In some examples, model configuration service 332 may use the following equation to calculate loss values.

$$ℒ ( y , \hat{y} ) = \frac{1}{n} {\sum }_{i = 1}^{n} \frac{1}{{m}^{′}} {\sum }_{j = 1}^{{m}^{′}} {( {y}_{ij} - {\hat{y}}_{ij} )}^{2} \qquad eq . 1$$

$${m}^{′} = \{ \begin{matrix}m - k , & i < n \\ m , & i = n\end{matrix}$$

Model configuration service 332 may be configured to calculate a loss [symbol] (y, ŷ) corresponding to each training dataset of unlabeled training data 356. In some examples, n represents a number of data segments in the sequence of overlapping data segments of the training dataset, m represents a length in data samples of each data segment, and k represents a size in data samples of a patch overlap corresponding to each data segment. In some examples, y is the true data sample amplitude corresponding to the training dataset and ŷ is a predicted sample amplitude corresponding to the predicted dataset generated using autoregression based on the training dataset.

Once model configuration service 332 normalizes training datasets of unlabeled training data 356, partitions the training datasets to create sequences of overlapping data segments, adds positional encoding to the sequences of overlapping data segments, uses autoregression to generate predicted datasets based on unlabeled training data 356, and calculates loss values, model configuration service 332 may train machine learning model 354 based on the unlabeled training data 356 that is processed during pretraining and based on labeled training data 358. That is, model configuration service 332 may pretrain machine learning model 354 by processing unlabeled training data 356 to gain information that is useful for training machine learning model 354, and model configuration service 332 may train machine learning model 354 using the unlabeled training data 356, information corresponding to unlabeled training data 356 acquired during pretraining, and the labeled training data 358 that already includes labels providing information corresponding to the labeled training data 358.

Data analyzer 330 may execute machine learning model 354 to perform one or more downstream tasks including classification tasks (e.g., AF classification or other arrythmia classification) and/or one or more segmentation tasks (e.g., identifying fiducials). Segmentation of cardiac signals may consist of locating and/or splitting a cardiac signal into constituent parts. For example, data analyzer 330 may locate P, Q, R, S, and T waves in cardiac data 352 by executing machine learning model 354. In some examples, data analyzer 330 uses machine learning model 354 to locate fiducials individually, or as unique objects. In some examples, data analyzer 330 uses machine learning model 354 to locate an interval or segment between the fiducials and uses post-processing to find individual fiducials.

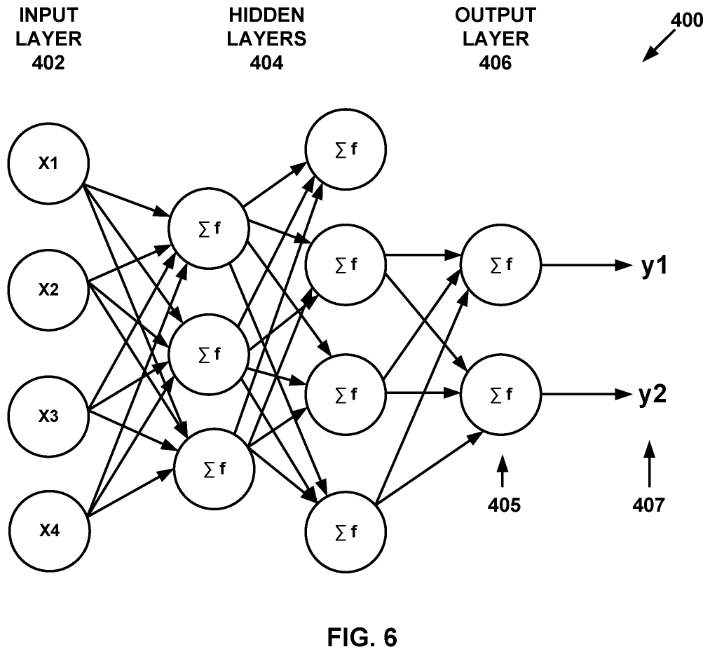

FIG. 6 is a conceptual diagram illustrating an example machine learning model 400 configured to output information corresponding to cardiac data, in accordance with one or more techniques of this disclosure. Machine learning model 400 is an example of a deep learning model, or deep learning algorithm. One or more of IMD 10, computing devices 12, or computing system 20 (e.g., model configuration service 272 and/or model configuration service 332) may train, store, and/or utilize machine learning model 400, but other devices may apply inputs associated with a particular patient to machine learning model 400 in other examples. Some non-limiting examples of machine learning techniques include Bayesian probability models, Hawkes processes, Support Vector Machines, K-Nearest Neighbor algorithms, and Multi-layer Perceptron.

As shown in the example of FIG. 6, machine learning model 400 may include include input layer 402, hidden layer 404, and output layer 406. Output layer 406 comprises the output from the transfer function 405 of output layer 406. Input layer 402 represents each of the input values X1 through X4 provided to machine learning model 400. The number of inputs may be less than or greater than 4, including much greater than 4, e.g., hundreds or thousands. In some examples, the input values may be parameters determined based on cardiac data 184, 292, 352, including those described herein, and in some cases other parameter data 182, 290, 350.

Each of the input values for each node in the input layer 402 is provided to each node of hidden layer 404. In the example of FIG. 6, hidden layers 404 include two layers, one layer having four nodes and the other layer having three nodes, but fewer or greater number of nodes may be used in other examples. Each input from input layer 402 is multiplied by a weight and then summed at each node of hidden layers 404. During training of machine learning model 400, the weights for each input are adjusted to establish the relationship between input physiological parameter values and one or more output values indicative of a health state of the patient. In some examples, one hidden layer may be incorporated into machine learning model 400, or three or more hidden layers may be incorporated into machine learning model 400, where each layer includes the same or different number of nodes.

The result of each node within hidden layers 404 is applied to the transfer function of output layer 406. The transfer function may be liner or non-linear, depending on the number of layers within machine learning model 400. Example non-linear transfer functions may be a sigmoid function or a rectifier function. The output 407 of the transfer function may be a value or values indicative a classification of an arrythmia such as AF or AV block or an indication of one or more fiducials in cardiac data. By applying the data to a machine learning model, such as machine learning model 400, processing circuitry of system 2 is able to determine the information corresponding to patient 4 with great specificity and sensitivity.

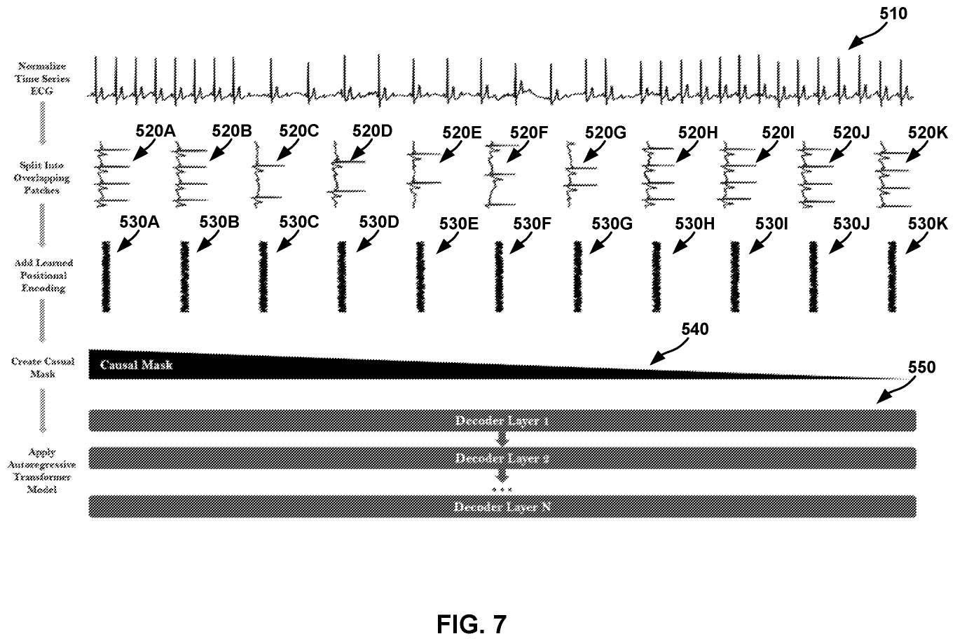

FIG. 7 is a conceptual diagram illustrating a process for pretraining unlabeled training data, in accordance with one or more techniques of this disclosure. As seen in FIG. 7, the process includes a training dataset 510, a sequence of overlapping data segments 520A-520K (collectively, “data segments 520”), learned positional encoding 530A-530K (collectively, “learned positional encoding 530”), causal mask 540, and transformer model 550.

In some examples, training dataset 510 may represent a training dataset of unlabeled training data 356. Model configuration service 332 may partition training dataset 510 into a sequence of overlapping data segments 520. In some examples, each data segment of data segments 520 may include one or more data samples that are also present in one or more other data segments of data segments 520. For example, data segment 520D may include one or more data samples that are also present in data segment 520C and one or more data samples that are also present in data segment 520E. In some examples, each data segment of data segments 520 may include one or more data samples that are not present in any other data samples of data segments 520. That is, each data segment of data segments 520 may include a portion that does not overlap with other data segments.

In some examples, model configuration service 332 may add learned positional encoding 530 to overlapping data segments 520. For example, model configuration service 332 may add learned positional encoding 530A to data segment 520A, model configuration service 332 may add learned positional encoding 530B to data segment 520B, model configuration service 332 may add learned positional encoding 530C to data segment 520C, and so on. Learned positional encoding 530 may indicate a position of each data segment of data segments 520 within training dataset 510 relative to other data segments of data segments 520. Causal mask 540 ensures that only previous portions of training dataset 510 are used to predict future portions of training dataset 510. Model configuration service 332 may execute transformer model 550 to process training dataset 510 to generate a predicted dataset. For example, model configuration service 332 may use transformer model 550 to generate a predicted dataset based on training dataset 510 using autoregression.

When model configuration service 332 generates a predicted dataset based on training dataset 510 using autoregression, model configuration service 332 may generate one or more loss values based on the predicted dataset and training dataset 510. For example, model configuration service 332 may generate a set of loss values that each indicate a difference between a non-overlapping portion of a data segment of data segments 520 and a corresponding portion of the predicted dataset generated using training dataset 510. In some examples, model configuration service 332 may generate a single loss value based on the set of loss values each corresponding to a data segment of data segments 520. That is, the single loss value may represent an accuracy at which model configuration service 332 generated a predicted dataset based on training dataset 510.

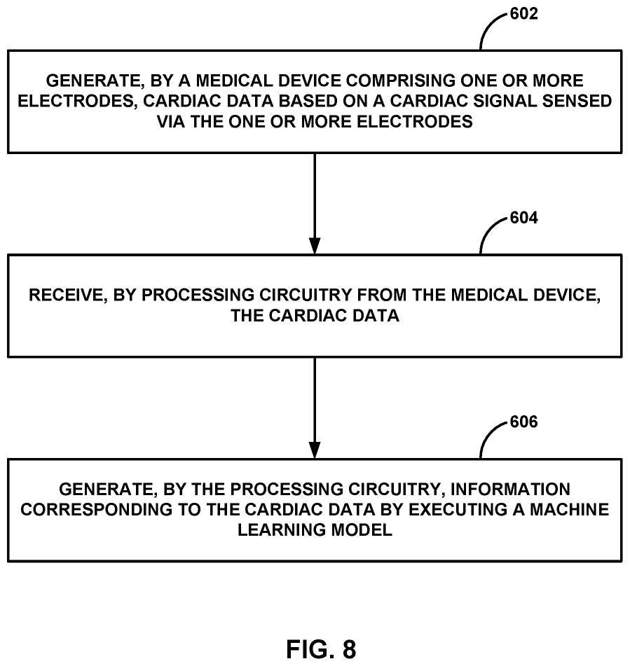

FIG. 8 is a flow diagram illustrating an example method for using a machine learning model to generate information corresponding to cardiac data collected from a patient, in accordance with one or more techniques of this disclosure. FIG. 8 is described with respect to medical device system 2 of FIG. 1. However, the techniques of FIG. 8 may be performed by different components of medical device system 2 or by additional or alternative medical device systems.

IMD 10 may generate cardiac data based on a cardiac signal sensed via one or more electrodes of IMD 10 (602). In some examples, the cardiac signal may represent an ECG signal or an EGM signal. Processing circuitry 22 of computing system 20 may receive the cardiac data from IMD 10 (604). Processing circuitry 22 may control HMS 26 to generate information corresponding to the cardiac data by executing a machine learning model (606). In some examples, the machine learning model is pretrained without supervision using a plurality of unlabeled training datasets. In some examples, the machine learning model is trained with supervision to identify the information corresponding to the cardiac data using a plurality of labeled training datasets.

The following numbered clauses may demonstrate one or more aspects of the disclosure.
Clause 1: A medical device system includes a medical device comprising one or more electrodes and configured to generate cardiac data based on a cardiac signal sensed via the one or more electrodes; and a memory configured to store: a machine learning model; a first plurality of training datasets, wherein each training dataset of the first plurality of training datasets not labeled with information corresponding to the dataset; and a second plurality of training datasets, wherein each training dataset of the second plurality of training datasets is labeled with information corresponding to the dataset. Additionally, the medical device system includes processing circuitry in communication with the memory, wherein the processing circuitry is configured to: receive, from the medical device, the cardiac data; and generate information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.
Clause 2: The medical device system of clause 1, wherein to generate information corresponding to the cardiac data, the processing circuitry is configured to identify one or more fiducials of the cardiac data.
Clause 3: The medical device system of clause 1, wherein to generate information corresponding to the cardiac data, the processing circuitry is configured to determine whether the cardiac data indicates atrial fibrillation (AF).
Clause 4: The medical device system of clause 1, wherein the processing circuitry is configured to pretrain the machine learning model by, for each training dataset of the first plurality of training datasets: performing autoregression to generate a predicted dataset corresponding to the training dataset; and comparing the predicted dataset with the training dataset.
Clause 5: The medical device system of clause 4, wherein to perform autoregression to generate the predicted dataset, the processing circuitry is configured to predict, at each time point of a sequence of time points of the training dataset, a future portion of the training dataset based on a portion of the training dataset occurring before the time point.
Clause 6: The medical device system of clause 4, wherein prior to performing autoregression, the processing circuitry is further configured to normalize each training dataset of the first plurality of training datasets by: subtracting, from each data sample of the training dataset, a mean data sample value corresponding to the training dataset; and dividing each data sample of the training dataset by a standard deviation data value corresponding to the training dataset.
Clause 7: The medical device system of clause 4, wherein the processing circuitry is further configured to pretrain the machine learning model by, for each training dataset of the first plurality of training datasets: partitioning the training dataset into a plurality of data segments, wherein each data segment of the plurality of data segments includes a set of data samples that are also present in one or more other data segments of the plurality of data segments such that the plurality of data segments form a sequence of overlapping data segments.
Clause 8: The medical device system of clause 7, wherein to compare the predicted dataset with the training dataset, the processing circuitry is configured to: calculate, for each data segment of the plurality of data segments of the training dataset, a loss value based on a difference between a non-overlapping portion of the data segment of the training dataset and a corresponding portion of the predicted dataset.
Clause 9: The medical device system of clause 7, wherein the processing circuitry is further configured to pretrain the machine learning model by, for each training dataset of the first plurality of training datasets: applying positional encoding to each data segment of the plurality of data segments corresponding to the training dataset, wherein the positional encoding applied to each data segment of the plurality of data segments includes information corresponding to a position of the data segment within the sequence of overlapping data segments.
Clause 10: The medical device system of clause 7, wherein the processing circuitry is further configured to: pretrain the machine learning model using the first plurality of training datasets; and train the pretrained machine learning model using the second plurality of training data sets to generate the machine learning model.
Clause 11: A method of operating a medical device system includes a medical device comprising one or more electrodes and configured to generate cardiac data based on a cardiac signal sensed via the one or more electrodes, the method comprising: receiving, by processing circuitry of the medical device system from the medical device, the cardiac data, wherein the processing circuitry is in communication with a memory of the medical device system. The memory is configured to store: a machine learning model; a first plurality of training datasets, wherein each training dataset of the first plurality of training datasets not labeled with information corresponding to the dataset; and a second plurality of training datasets, wherein each training dataset of the second plurality of training datasets is labeled with information corresponding to the dataset. Additionally, the method comprises generating, by the processing circuitry, information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.
Clause 12: The method of clause 11, wherein generating information corresponding to the cardiac data comprises identifying, by the processing circuitry, one or more fiducials of the cardiac data.
Clause 13: The method of clause 11, wherein generating information corresponding to the cardiac data comprises determining, by the processing circuitry, whether the cardiac data indicates atrial fibrillation (AF).
Clause 14: The method of clause 11, wherein the method further comprises pretraining, by the processing circuitry, the machine learning model by, for each training dataset of the first plurality of training datasets: performing autoregression to generate a predicted dataset corresponding to the training dataset; and comparing the predicted dataset with the training dataset.
Clause 15: The method of clause 14, wherein performing autoregression to generate the predicted dataset comprises predicting, by the processing circuitry at each time point of a sequence of time points of the training dataset, a future portion of the training dataset based on a portion of the training dataset occurring before the time point.
Clause 16: The method of clause 14, wherein prior to performing autoregression, the method further comprises normalizing, by the processing circuitry, each training dataset of the first plurality of training datasets by: subtracting, from each data sample of the training dataset, a mean data sample value corresponding to the training dataset; and dividing each data sample of the training dataset by a standard deviation data sample value corresponding to the training dataset.
Clause 17: The method of clause 14, further comprising pretraining, by the processing circuitry, the machine learning model by, for each training dataset of the first plurality of training datasets: partitioning the training dataset into a plurality of data segments, wherein each data segment of the plurality of data segments includes a set of data samples that are also present in one or more other data segments of the plurality of data segments such that the plurality of data segments form a sequence of overlapping data segments.
Clause 18: The method of clause 17, wherein comparing the predicted dataset with the training dataset comprises: calculating, by the processing circuitry for each data segment of the plurality of data segments of the training dataset, a loss value based on a difference between a non-overlapping portion of the data segment of the training dataset and a corresponding portion of the predicted dataset.
Clause 19: The method of clause 11, further comprising: pretraining, by the processing circuitry, the machine learning model using the first plurality of training datasets; and training, by the processing circuitry, the pretrained machine learning model using the second plurality of training data sets to generate the machine learning model.
Clause 20: A non-transitory computer-readable storage medium includes program instructions that, when executed by processing circuitry of a medical device system comprising one or more electrodes and configured to generate cardiac data based on a cardiac signal sensed via the one or more electrodes, cause the processing circuitry to: receive the cardiac data, wherein the processing circuitry is in communication with a memory of the medical device system. The memory is configured to store: a machine learning model; a first plurality of training datasets, wherein each training dataset of the first plurality of training datasets not labeled with information corresponding to the dataset; and a second plurality of training datasets, wherein each training dataset of the second plurality of training datasets is labeled with information corresponding to the dataset. Additionally, the instructions cause the processing circuitry to generate information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1. A medical device system comprising:
a medical device comprising one or more electrodes and configured to generate cardiac data based on a cardiac signal sensed via the one or more electrodes; and
a memory configured to store:
a machine learning model;
a first plurality of training datasets, wherein each training dataset of the first plurality of training datasets not labeled with information corresponding to the dataset; and
a second plurality of training datasets, wherein each training dataset of the second plurality of training datasets is labeled with information corresponding to the dataset; and
processing circuitry in communication with the memory, wherein the processing circuitry is configured to:
receive, from the medical device, the cardiac data; and
generate information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.

2. The medical device system of claim 1, wherein to generate information corresponding to the cardiac data, the processing circuitry is configured to identify one or more fiducials of the cardiac data.

3. The medical device system of claim 1, wherein to generate information corresponding to the cardiac data, the processing circuitry is configured to determine whether the cardiac data indicates atrial fibrillation (AF).

4. The medical device system of claim 1, wherein the processing circuitry is configured to pretrain the machine learning model by, for each training dataset of the first plurality of training datasets:
performing autoregression to generate a predicted dataset corresponding to the training dataset; and
comparing the predicted dataset with the training dataset.

5. The medical device system of claim 4, wherein to perform autoregression to generate the predicted dataset, the processing circuitry is configured to predict, at each time point of a sequence of time points of the training dataset, a future portion of the training dataset based on a portion of the training dataset occurring before the time point.

6. The medical device system of claim 4, wherein prior to performing autoregression, the processing circuitry is further configured to normalize each training dataset of the first plurality of training datasets by:
subtracting, from each data sample of the training dataset, a mean data sample value corresponding to the training dataset; and
dividing each data sample of the training dataset by a standard deviation data sample value corresponding to the training dataset.

7. The medical device system of claim 4, wherein the processing circuitry is further configured to pretrain the machine learning model by, for each training dataset of the first plurality of training datasets:
partitioning the training dataset into a plurality of data segments, wherein each data segment of the plurality of data segments includes a set of data samples that are also present in one or more other data segments of the plurality of data segments such that the plurality of data segments forms a sequence of overlapping data segments.

8. The medical device system of claim 7, wherein to compare the predicted dataset with the training dataset, the processing circuitry is configured to:
calculate, for each data segment of the plurality of data segments of the training dataset, a loss value based on a difference between a non-overlapping portion of the data segment of the training dataset and a corresponding portion of the predicted dataset.

9. The medical device system of claim 7, wherein the processing circuitry is further configured to pretrain the machine learning model by, for each training dataset of the first plurality of training datasets:
applying positional encoding to each data segment of the plurality of data segments corresponding to the training dataset,
wherein the positional encoding applied to each data segment of the plurality of data segments includes information corresponding to a position of the data segment within the sequence of overlapping data segments.

10. The medical device system of claim 1, wherein the processing circuitry is further configured to:
pretrain the machine learning model using the first plurality of training datasets; and
train the pretrained machine learning model using the second plurality of training data sets to generate the machine learning model.

11. A method of operating a medical device system comprising a medical device comprising one or more electrodes and configured to generate cardiac data based on a cardiac signal sensed via the one or more electrodes, the method comprising:
receiving, by processing circuitry of the medical device system from the medical device, the cardiac data, wherein the processing circuitry is in communication with a memory of the medical device system, the memory configured to store:
a machine learning model;
a first plurality of training datasets, wherein each training dataset of the first plurality of training datasets not labeled with information corresponding to the dataset; and
a second plurality of training datasets, wherein each training dataset of the second plurality of training datasets is labeled with information corresponding to the dataset; and
generating, by the processing circuitry, information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.

12. The method of claim 11, wherein generating information corresponding to the cardiac data comprises identifying, by the processing circuitry, one or more fiducials of the cardiac data.

13. The method of claim 11, wherein generating information corresponding to the cardiac data comprises determining, by the processing circuitry, whether the cardiac data indicates atrial fibrillation (AF).

14. The method of claim 11, wherein the method further comprises pretraining, by the processing circuitry, the machine learning model by, for each training dataset of the first plurality of training datasets:
performing autoregression to generate a predicted dataset corresponding to the training dataset; and
comparing the predicted dataset with the training dataset.

15. A non-transitory computer-readable storage medium comprising program instructions that, when executed by processing circuitry of a medical device system comprising one or more electrodes and configured to generate cardiac data based on a cardiac signal sensed via the one or more electrodes, cause the processing circuitry to: receive the cardiac data, wherein the processing circuitry is in communication with a memory of the medical device system, the memory configured to store:
a machine learning model;
a first plurality of training datasets, wherein each training dataset of the first plurality of training datasets not labeled with information corresponding to the dataset; and
a second plurality of training datasets, wherein each training dataset of the second plurality of training datasets is labeled with information corresponding to the dataset; and
generate information corresponding to the cardiac data by executing the machine learning model, wherein the machine learning model is pretrained without supervision using the first plurality of training datasets, and wherein the machine learning model is trained with supervision to identify the information corresponding to the cardiac data using the second plurality of training datasets.

16. The method of claim 14, wherein performing autoregression to generate the predicted dataset comprises predicting, by the processing circuitry at each time point of a sequence of time points of the training dataset, a future portion of the training dataset based on a portion of the training dataset occurring before the time point.

17. The method of claim 14, wherein prior to performing autoregression, the method further comprises normalizing, by the processing circuitry, each training dataset of the first plurality of training datasets by:
subtracting, from each data sample of the training dataset, a mean data sample value corresponding to the training dataset; and
dividing each data sample of the training dataset by a standard deviation data sample value corresponding to the training dataset.

18. The method of claim 14, further comprising pretraining, by the processing circuitry, the machine learning model by, for each training dataset of the first plurality of training datasets:
partitioning the training dataset into a plurality of data segments, wherein each data segment of the plurality of data segments includes a set of data samples that are also present in one or more other data segments of the plurality of data segments such that the plurality of data segments form a sequence of overlapping data segments.

19. The method of claim 18, wherein comparing the predicted dataset with the training dataset comprises:
calculating, by the processing circuitry for each data segment of the plurality of data segments of the training dataset, a loss value based on a difference between a non-overlapping portion of the data segment of the training dataset and a corresponding portion of the predicted dataset.

20. The method of claim 11, further comprising:
pretraining, by the processing circuitry, the machine learning model using the first plurality of training datasets; and
training, by the processing circuitry, the pretrained machine learning model using the second plurality of training data sets to generate the machine learning model.
